(19) United States
(12) Patent Application Publication
(10) Pub. No.: US 2003/0170814 A1
(43) Pub. Date: Sep. 11, 2003

(54) Systems and methods for providing runtime execution of discovery logic from biological and chemical data

(76) Inventors: Sermet Yucel, Edina, MN (US); Maria Germana Paterlini, Edina, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/354,930

(22) Filed: Jan. 29, 2003

Related U.S. Application Data
(63) Continuation-in-part of application No. 10/034,601, filed on Dec. 26, 2001.
(60) Provisional application No. 60/352,729, filed on Jan. 29, 2002.

(51) Int. Cl. C12P 21/06

(52) U.S. Cl. 435/69.1

(57) ABSTRACT

A method, apparatus, and program products for designing, implementing, distributing and deploying computer programs. Such programs bind the symbolic representation of a biological or chemical process to a physical implementation in computer memory. A Knowledge model defines a model for representing biological or chemical entities, knowledge on these systems, and packaging facts and intelligence using Knowledge Oriented Programming (KOP). The resulting knowledge components are implemented as off the shelf object oriented programming languages and tools. A Logic Model interprets existing algorithms and computational tools according to KOP. It also provides tools for encoding inference about the system. The Discovery Model assembles the components of the Knowledge and Logic Model for execution in computer memory. The Graphical User Interface (GUI) provides a tool for designing and executing a discovery application according to KOP.

### CROSS REFERENCE TO RELATED APPLICATIONS

This application claims priority to U.S. provisional application serial No. 60/352,729, filed Jan. 29, 2002, entitled BIOINFORMATICS KNOWLEDGE ORIENTED PROGRAMMING, which is hereby incorporated by reference. This application is a continuation-in-part of and claims priority to U.S. patent application Ser. No. 10/034,601, filed Dec. 26, 2001, entitled KNOWLEDGE ORIENTED PROGRAMMING, which is hereby incorporated by reference.

### LIMITED COPYRIGHT WAIVER

A portion of the disclosure of this patent document contains material to which the claim of copyright protection is made. The copyright owner has no objection to the facsimile reproduction by any person of the patent document or the patent disclosure, as it appears in the U.S. Patent and Trademark Office file or records, but reserves all other rights whatsoever.

### FIELD OF THE INVENTION

The invention relates generally to computer systems, and more particularly to a systems and methods that use knowledge oriented programming to execute on a computer the logical process of discovery from biological and/or chemical data.

### BACKGROUND

Informatics for the Life Sciences is a science that provides information on the function of the human system through the use of methods, apparatus, and programs that extract information from genomics, proteonomics, and chemical data.

The wealth of information arising from high-throughput genomics, proteonomics, and combinatorial chemistry presents a challenge to academia, pharmaceutical, and biotechnology companies. The discovery process requires complex data analysis to derive new knowledge and the application of algorithms to model biological systems.

Current bioinformatics and computational biotechnology tools focus on developing and improving specific algorithms to develop a method for extracting information. Automated analysis of complex biological systems typically requires the integrated power of three distinct technologies: Relational Databases, Logic Computing, and Object Oriented Programming. These three methodologies, when used separately, may not always provide what users need. The computational power of relational databases queries is limited by the expressive power of query languages, such as Structure Query Language (SQL). SQL becomes rapidly ineffective when complex objects must be represented, correlated, and analyzed with powerful algorithms. Artificial Intelligence tools, such as Rule engines, deductive databases, and expert systems can represent and execute complex knowledge-based queries, but Life Sciences tools based on these technologies are typically specialized solutions that are not likely to be available as easy-to-use and economical commercial products. Object oriented programming is best suited to implement algorithms, interactive user interfaces, and visualization tools.

What is needed is a common, easy-to-use automation framework by which disparate data can be correlated, transformed into knowledge, used in algorithmic computations, and shared among diverse groups of researchers.

### SUMMARY

In various embodiments, a method, system, apparatus, and signal-bearing medium are provided for designing, implementing, distributing, and deploying computer programs that consist of packaged knowledge components for applications in the life sciences written in object oriented programming languages and modeled according to knowledge oriented programming (KOP). A discovery model for application in the Life Sciences defines a model for representing facts, intelligence, and packaging facts and intelligence into readily usable knowledge components implemented in off-the-shelf object-oriented programming languages and tools. Components of the discovery process are provided that can be executed by a KOP kernel. A KOP runtime is provided to bind symbols and execute computer programs made of generic components designed according to the KOP environment. A user interface, accessible via the Internet or other media, is easily customizable by the user.

### BRIEF DESCRIPTION OF THE FIGURES

FIG. 1 depicts a block diagram of the encoding of the discovery process for life sciences applications, according to an embodiment of the invention.

FIG. 2 depicts a block diagram of example encoding of entities into the Knowledge Model, according to an embodiment of the invention.

FIG. 3 depicts a block diagram of an example of a biological component, a protein, modeled using Knowledge Oriented Programming, according to an embodiment of the invention.

FIG. 4 depicts a block diagram of an example of components of the Logic Model, according to an embodiment of the invention.

FIG. 5 depicts a block diagram of an example of how an algorithm is used in the Logic Model, according to an embodiment of the invention.

FIG. 6 depicts a block diagram of an example of a Discovery Model, according to an embodiment of the invention.

FIG. 7 depicts an example of Graphical User Interface for the discovery application that uses a KOP environment, according to an embodiment of the invention.

FIG. 8 depicts a block diagram of an example system for implementing an embodiment of the invention.

### DETAILED DESCRIPTION

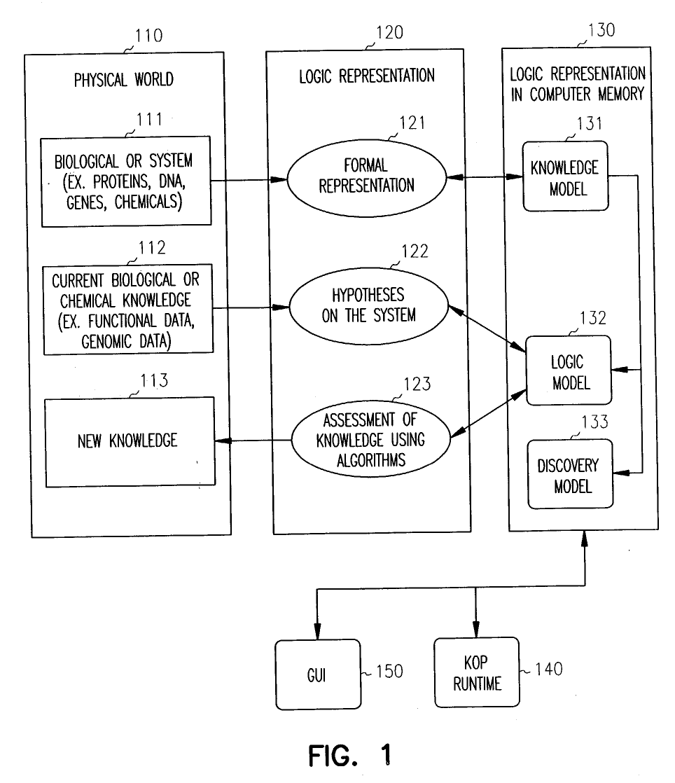

FIG. 1 depicts a schematic diagram illustrating the interaction between data and software, including GUI 150 and runtime 140 according to an embodiment of the invention. The computer memory representation illustrated in block 130 is better understood by first describing blocks 110 and 120. The Physical World 110 comprises the biological/chemical system 111 under consideration and the set of entities that describe this system. Examples of such entities are genes, proteins and chemicals, although in other embodiments any appropriate entities may be used.

Current Biological Knowledge 112 comprises the set of findings about a particular biological or chemical system. Such findings may be obtained through experimentation or computation of the biological or chemical variables. Examples of such finding are the discovery of a protein function, the mapping of a metabolic pathway, or relation between protein families. Current Biological/Chemical Knowledge 112 represents the state of knowledge for a biological or chemical system at a given time.

New Knowledge 113 comprises the discovery of new relations among biological or chemical entities. The new discoveries may validate or refute current hypothesis on the system. New knowledge 113 can be obtained through various means, such as statistical analysis, logical analysis, and computations.

Logic Representation 120 is a human interpretation of the Physical World 110. Entities of the biological or chemical system 111 are in various embodiments stored in databases, text files, and may be recorded in a variety of paper or electronic media, although in other embodiments any type of storage may be used. Hypotheses on the System 122 and assessment of current knowledge 123 are formulated based on processes chosen to analyze the data in the formal representation 121. In various embodiments, such processes can be computational algorithms, statistical analyses, or inference tools, although any appropriate processes may be used. In various embodiments, processes can be expressed in computer programs, notebook notes, or speech, in other embodiments any appropriate expression may be used. Representation in Computer Memory 130 is the capture of the Logic Representation 120 of the Physical World 110 using a Knowledge Oriented Programming (KOP) formalism. Knowledge Oriented Programming comprises an environment that transforms data into knowledge and utilizes it in the discovery process. In some embodiments, the KOP environment is a computing environment that integrates object-oriented programming, first order logic, and relations of complex objects. The KOP environment provides mechanisms that support the design, implementation, distribution, and deployment of computer programs that are comprised of packaged knowledge components written in object oriented programming languages. The KOP environment used in some embodiments of the invention is described in U.S. patent application Ser. No. 10/034,601, filed Dec. 26, 2001, entitled KNOWLEDGE ORIENTED PROGRAMMING, which is hereby incorporated by reference herein for all purposes.

The Knowledge Model 131 comprises a set of one or more components for declaring and storing the variables of the Biological or Chemical system 111. The components may include one or more methods that may involve accessing databases, text files, or other types of recorded material to populate the component of the Knowledge Model 131. The Logic Model 132 comprises a method for accessing existing algorithms and statistical tools (i.e. computational tools). The Logic Model 132 provides the language for interpreting the existing computational tools according to Knowledge Oriented Programming. The Logic Model 132 further provides a method for storing inference data, such as rules and constraints, which in some embodiments are expressed according to a KOP formalism. The Knowledge Model 131 and Logic Model 132 are assembled for execution by the Discovery Model 133. The Discovery Model 133 executes the discovery logic in computer memory by using the KOP runtime. In some embodiments, code representing models 131, 132 and 133 is generated in the following fashion. The user provides the logical declaration of an entity (for example, the entity Protein) through a Graphical User Interface (GUI) 150 and/or an application descriptor language that may be part of the GUI 150 or separately provided. In some embodiments, such GUI will have a text editor for declaring the properties of the entity (for example, a user may define a Protein as an entity with properties such as sequence, structure, etc.). In further alternative embodiments, the GUI 150 includes graphical elements such as menus, buttons, and icons that may be used to declare property elements. The user declaration is then automatically converted into one or more components that may be stored and executed in computer memory using the KOP runtime 140 without additional intervention from the user.

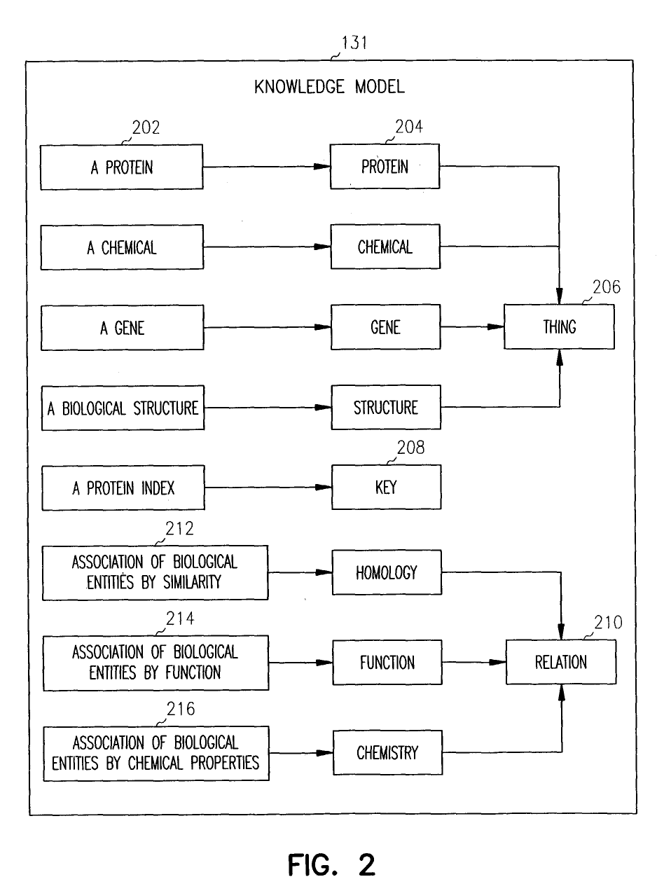

FIG. 2 depicts a block diagram of the encoding of the entities of the Biological/Chemical System 111 into the Knowledge Model 131. For example, a protein 202 is represented by a Protein type 204. This type may be encoded using an object oriented language. At the Meta Model level, the Protein type 204 is identified as type Thing 206, which is an element of Knowledge Oriented Programming environment. An identification number (ID #) used to identify biological or chemical entities in databases may be specified using the Key type 208 of a KOP environment. Relationships among biological or chemical entities may be represented at the Meta Model level by the Relation type 210, which may be an element defined by the KOP environment. Such Relation types are then implemented using an object oriented language. Examples of a relationship are association by similarity 214 (such as structure similarity or sequence similarity), association by function 214 (for example proteins that belong to the same metabolic pathway), or by chemical characteristics 216 (for example classes of chemical compounds).

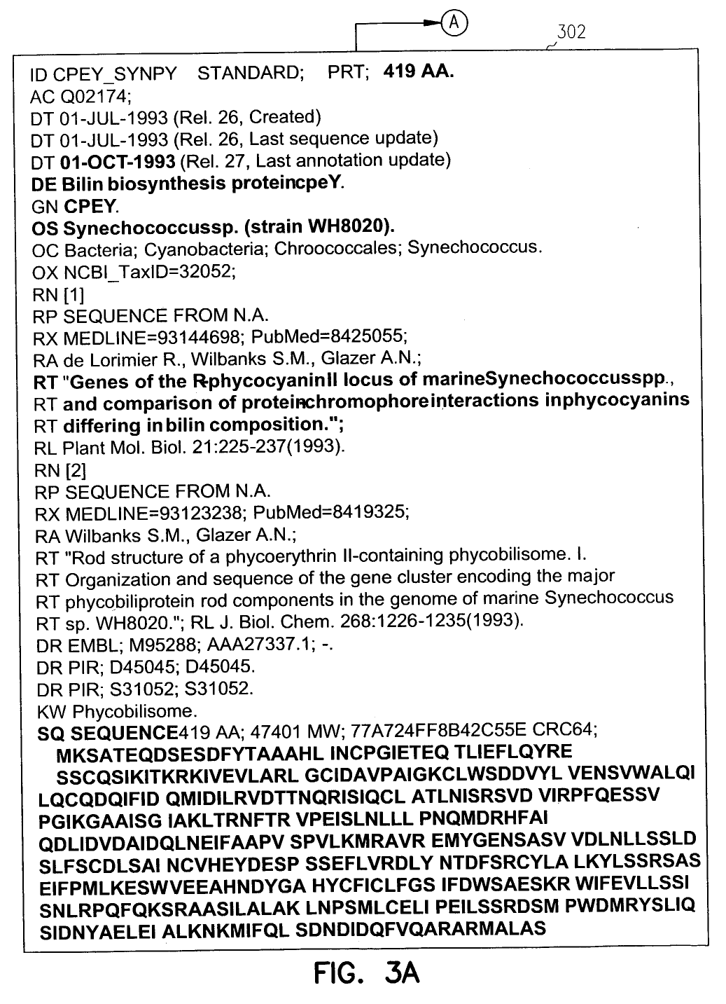

FIG. 3 depicts an example of how biological and chemical entities are transformed into computer code using a KOP runtime. Within Knowledge Model 131, a user describes a biological or chemical entity through a GUI 150. Block 302 shows a typical database record for a protein. A protein has properties including a sequence, organism of origin, physiological function, etc. (outlined in bold characters in block 302). The user may use the GUI to specify such properties by inputting records such as “protein_id”, “date”, “name”, etc. By using the KOP-run time 140, such records are converted into KOP types and then into computer code. The entity Protein is now of type Thing and it is implemented as a class (“public class Protein extends Thing” 206). The class Protein extends the class Thing, which, in turn, is the implementation of the type Thing of the KOP environment. The “protein_id” is associated with Key 208 and it is obtained using a getKey method. Additional properties may be defined using additional methods provided within the KOP runtime environment. For example, “getSequence” returns the sequence of the protein. Following generation of Protein as a Thing, components that extend Fact and Relation defined by the KOP environment are also generated. The example shows an implementation using Java, but the process is applicable to other object oriented languages as well. For example, the C++ language could be used. Computer code 304 is generated automatically without user intervention. Properties may be added, or deleted as needed, and the code may be regenerated to reflect the modifications to the various models.

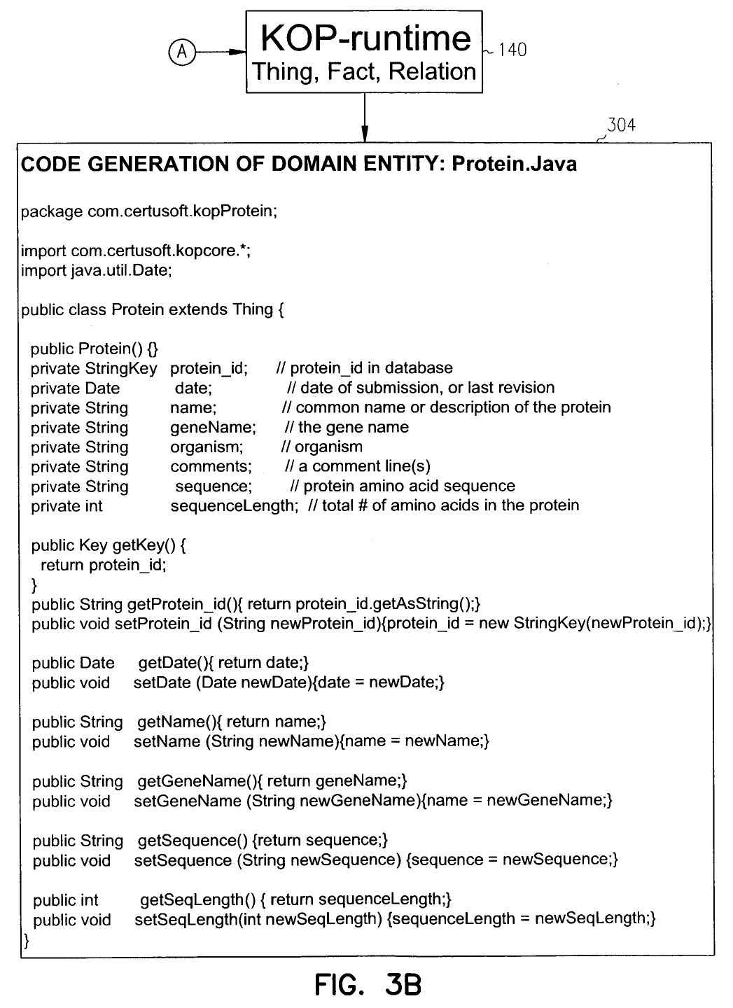

FIG. 4 depicts a block diagram of the encoding of the Logic Model 132. Computational operations among biological or chemical entities are usually carried out using computational chemistry tools, bioinformatics tools (such as algorithms for sequence comparison or pattern search), and inference tools. Such inference tools may be external or internal algorithms or may be logical statements expressed by the user of the system using GUI 150. Examples are computational chemistry software 402, bioinformatics algorithms 404, and validation and inference tools 406. Such components are represented in the Logic Model 132 by using the Relation Function 408 elements of the Meta model and described using the KOP environment. Wrappers 410, 412, and 414 translate the input and output of the algorithms into components defined and understood by the KOP environment. Wrappers 410, 412, and 414 comprise computer code written using an object oriented language. In an embodiment, a wrapper is a small program that translates the input and output from an existing program into elements of the KOP environment. Inputs and outputs to and from the wrapper can be defined using the GUI 150, while the user may supply the algorithm executed by the wrapper. Execution of the Relation Function is controlled by a Event/Event Handler pair of the Meta Model.

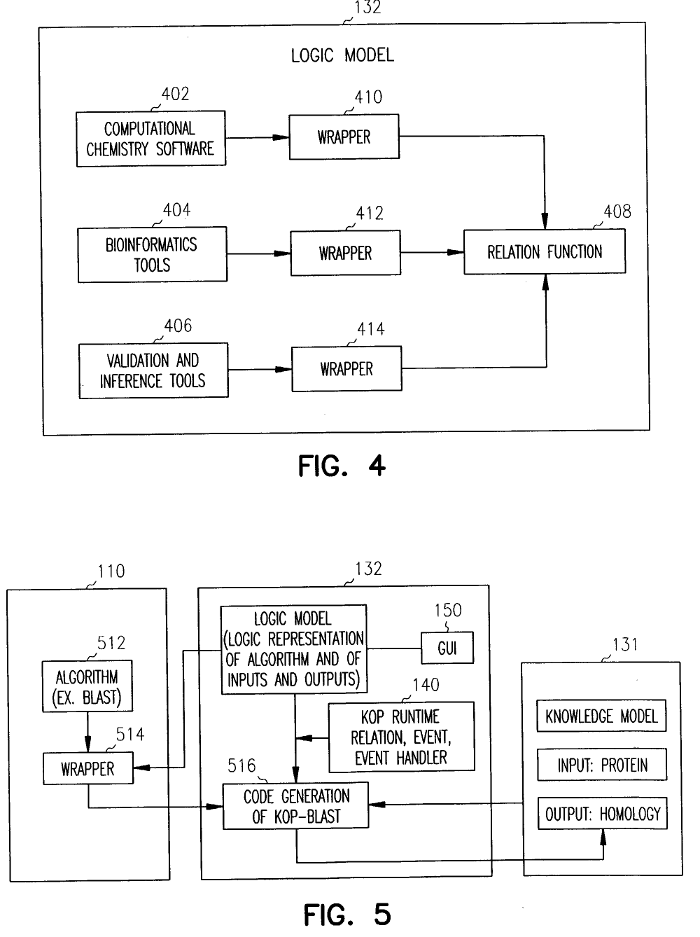

FIG. 5 depicts an example of a component of the Logic Model 132. In this example, the Logic Model 132 comprises an algorithm to compute sequence similarity between a list of proteins and a chosen protein. In this example, the BLAST (Basic Local Analysis Search Tool) algorithm 512 is used and a wrapper 514 is written to communicate with the components of the Knowledge Model 131. The BLAST algorithm is further described in Altschul S F, Gish W, Miller W, Myers E W, Lipman D J. BLAST Basic local alignment search tool. J Mol Biol. 215:403-410 (1990). The user, using the GUI 150, specifies the required inputs and outputs to BLAST. In this particular example, two inputs are required, the test protein and a protein database. These elements are described in the Knowledge Model by the type Protein 204. The output of the calculation will be a new relation that reports the degree of similarity of the test protein. The results are stored in a new entity, also designed according to the procedure in FIGS. 2 and 3. “KOP-BLAST” comprises generated computer code 516 which extends the Relation Function component of the KOP environment 140 and is handled at execution through an Event/EventHandler pair of the Meta Model. KOP-BLAST accepts elements of the Knowledge Model 131 and makes use of a wrapper 514. The class may be generated automatically, without user intervention.

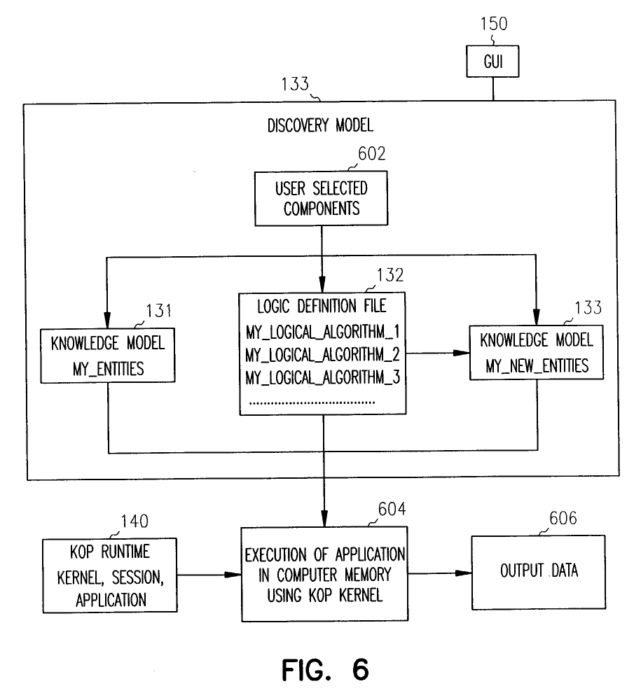

FIG. 6 depicts a block diagram of the design and execution of a discovery application using the Discovery Model 133. By using the GUI 150, a user selects the components 602 needed to execute the application. A discovery process may have any number of components, both in the Knowledge Model 131 and in the Logic Model 132. Components of the Knowledge Model are generated as described in FIG. 2. Various Logical Algorithms, generated as described in FIG. 5, can be cascaded as needed. The user designs the entire applications by specifying how different components are linked to each other. After assembly using the GUI 150, an Application 604 is executed in computer memory by the KOP runtime 140. The Discovery Model 133 makes use of Kernel, Application and Session of the Meta Model. Output Data 606 resulting from the execution of the application will be stored in entities described according to the Knowledge Model 131.

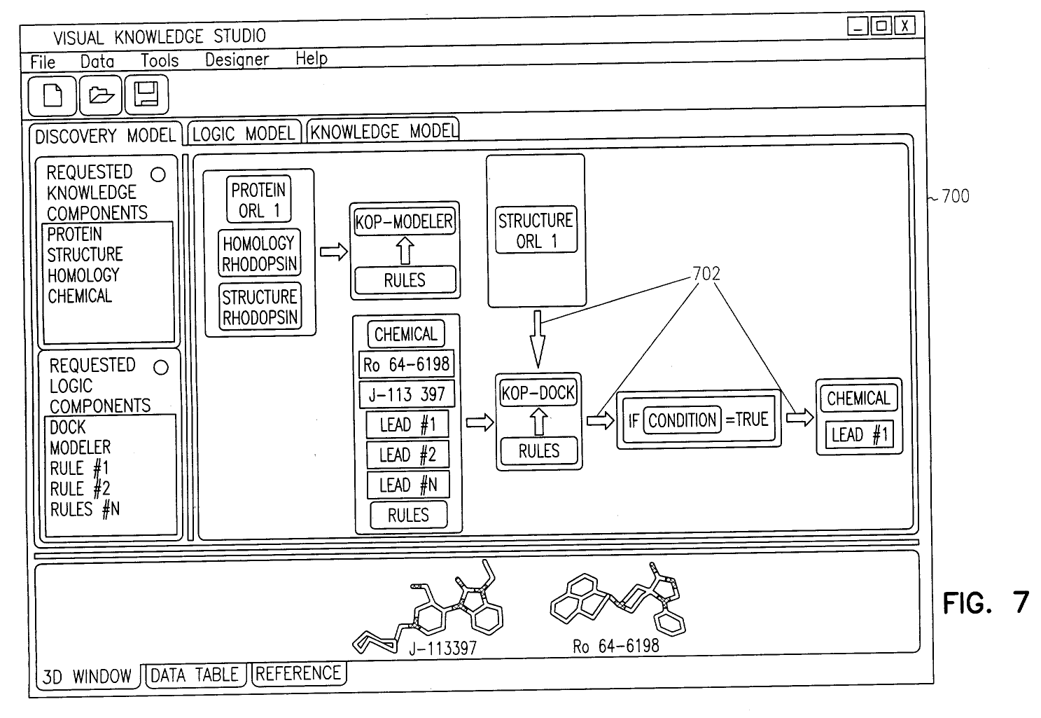

FIG. 7 is a block diagram of a screen image 700 of graphical user interface (GUI) 150 according to an embodiment of the invention that depicts how biological and chemical knowledge components, modeled according to a KOP environment are assembled and used by the user. The GUI 150 may be used to access, store, retrieve, design and to share the components of the Discovery Model 133. In some embodiments, the GUI 150 provides folders for the Knowledge Model 131, the Logic Model 132, and the Discovery Model 133. In some embodiments, a user first designs the components of the Knowledge Model 131 and the Logic Model 132. These are then accessed through a pull-down menu and imported into the Discovery Model 133 upon request.

In the example, the requested Knowledge Model 131 components are entities describing a Protein, its Structure, a Homology Relation and the chemicals to be tested (labeled Chemical in screen image 700). Logic Model 132 components are the functions and algorithms that may be used to carry out this test. In this example, algorithms such as DOCK, and MODELER may be called, as well as rules set up by the user (Rule #1 etc). The DOCK algorithm is further described in Meng, E. C., Shoichet, B. K., and Kuntz I. D. DOCK Automated docking with grid-based energy evaluation. J. Comp. Chem 13:505-524 (1992). The MODELER algorithm is further described in Fiser A, Sali A. MODELLER: generation and refinement of homology models. In: Methods in Enzymology. Ed: Carter, C. W. and Sweet, R. M. Academic Press, San Diego, Calif., 2001.

The components may then be presented on the Knowledge Model 131 using a drag and drop procedure. In the example, the Structure of a Protein (indicated here as ORL1) is obtained through a modeling procedure that involves finding a Homology relation with a known protein (rhodopsin) and then applying a modeling algorithm (KOP-Modeler). The modeled protein is then used in docking simulations (by invoking KOP-DOCK) and applying rules to screen the compounds. The end result is a list of chemical leads, e.g., a drug target, for the protein ORL1. In some embodiments, the arrows 702 represent a set of Event/Event Handler pairs that organize the logical flow of the application and execute it by binding it to the KOP-Kernel. The design flow is completely customizable, as the user may assemble the application as needed.

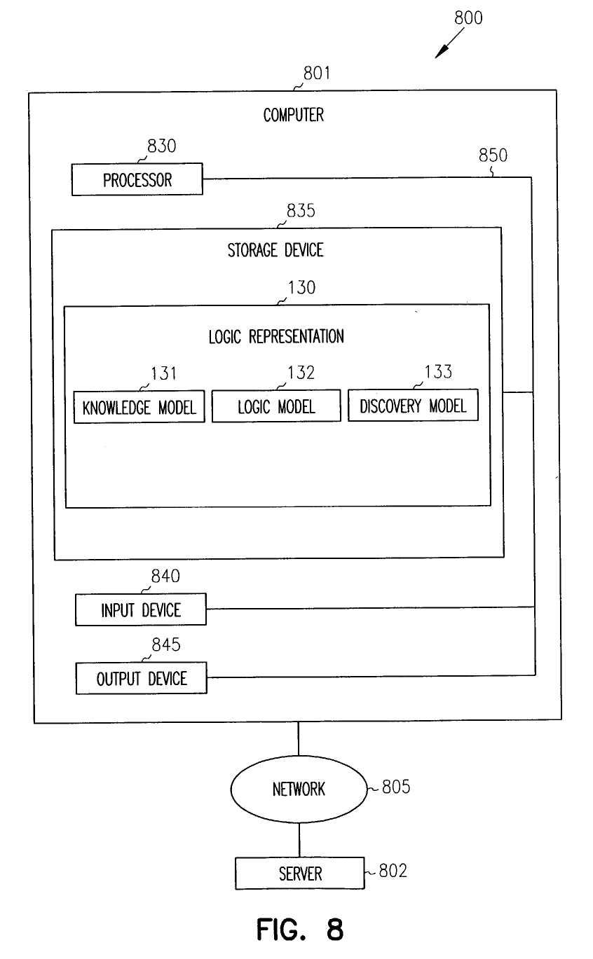

FIG. 8 depicts a block diagram of an example system 800 for implementing an embodiment of the invention. The system 800 includes a computer 801 connected to a server 802 via a network 805. Although one computer 801, one server 802, and one network 805 are shown, in other embodiments any number or combinations of them are present.

The computer 801 includes a processor 830, a storage device 835, an input device 840, and an output device 845, all connected directly or indirectly via a bus 850.

The processor 830 represents a central processing unit of any type of architecture, such as a CISC (Complex Instruction Set Computing), RISC (Reduced Instruction Set Computing), VLIW (Very Long Instruction Word), or hybrid architecture, although any appropriate processor may be used. The processor 830 executes instructions and includes that portion of the computer 801 that controls the operation of the entire computer. Although not depicted in FIG. 8, the processor 830 typically includes a control unit that organizes data and program storage in memory and transfers data and other information between the various parts of the computer 801. The processor 830 receives input data from the network 805 and the input device 840, reads and stores code and data in the storage device 835, and presents data to the network 805 and/or the output device 845.

Although the computer 801 is shown to contain only a single processor 830 and a single bus 850, the present invention applies equally to computers that may have multiple processors and to computers that may have multiple buses with some or all performing different functions in different ways.

The storage device 835 represents one or more mechanisms for storing data. For example, the storage device 835 may include read only memory (ROM), random access memory (RAM), magnetic disk storage media, optical storage media, flash memory devices, and/or other machine-readable media. In other embodiments, any appropriate type of storage device may be used. Although only one storage device 835 is shown, multiple storage devices and multiple types of storage devices may be present. Further, although the computer 801 is drawn to contain the storage device 835, it may be distributed across other electronic devices.

The storage device 835 includes the Logic Representation 130, which includes Knowledge Model 131, the Logic Model 132, and the Discovery Model 133, all of which include data and/or instructions capable of being executed on the processor 830 to carry out the functions of the present invention, as previously described above with reference to FIGS. 1-7. In another embodiment, some or all of the functions of the present invention are carried out via hardware. Of course, the storage device 835 may also contain additional software and data (not shown), which is not necessary to understanding the invention.

Although the Knowledge Model 131, the Logic Model 132, and the Discovery Model 133 are shown to be within the storage device 835 in the computer 801, in another embodiment they may be distributed across other systems, e.g., on the server 802 and accessed remotely.

The bus 850 may represent one or more busses, e.g., PCI, ISA (Industry Standard Architecture), X-Bus, EISA (Extended Industry Standard Architecture), or any other appropriate bus and/or bridge (also called a bus controller).

The computer 801 may be implemented using any suitable hardware and/or software, such as a personal computer or other electronic computing device. Portable computers, laptop or notebook computers, PDAs (Personal Digital Assistants), pocket computers, telephones, and mainframe computers are examples of other possible configurations of the computer 801. The hardware and software depicted in FIG. 8 may vary for specific applications and may include more or fewer elements than those depicted. For example, other peripheral devices such as audio adapters, or chip programming devices, such as EPROM (Erasable Programmable Read-Only Memory) programming devices may be used in addition to or in place of the hardware already depicted.

The server 802 may include components analogous to some or all of the components already described for the computer 801. In another embodiment, the server is not present.

The network 805 may be any type of network or combination of networks suitable for communicating between the computer 801 and the server 802. In another embodiment, the network 805 is not present.

As was described in detail above, aspects of an embodiment pertain to specific apparatus and method elements implementable on a computer or other electronic device. In another embodiment, the invention may be implemented as a program product for use with an electronic device. The programs defining the functions of this embodiment may be delivered to a computer via a variety of signal-bearing media, which include, but are not limited to:

(1) information permanently stored on a non-rewriteable storage medium, e.g., a read-only memory device attached to or within an electronic device, such as a CD-ROM readable by a CD-ROM drive;

(2) alterable information stored on a rewriteable storage medium, e.g., a hard disk drive or diskette; or

(3) information conveyed to a computer by a communications medium, such as through a computer or a telephone network, including wireless communications.

Such signal-bearing media, when carrying machine-readable instructions that direct the functions of the present invention, represent embodiments of the present invention.

Various embodiments of the present invention provide a method for extracting knowledge from biological and chemical data using a KOP environment. The KOP framework helps users rapidly process large biological and/or chemical data into knowledge. Application of a KOP environment to the biological and chemical sciences may meet the needs for facilitating and speeding discovery processes, such as drug design, although it may be used in any appropriate discovery process. Implementation of the method into software tools result in a system that can be customized by the end user, typically without the need of additional skills. The discovery process can be saved as a text file and easily shared among researchers. Security may be provided for defining ownership and access privileges. Some embodiments of the invention provide a system that can be accessed through a Web-accessible browser or that be started remotely (such as WebStart). Other embodiments are used on a stand-alone computer.

What is claimed is:

1. A signal-bearing medium bearing a model for building discovery logic, the model comprising:
a knowledge model comprising a specification of how a biological or a chemical entity is represented;
a logic model comprising a specification of how a set of one or more algorithms associated with the biological or chemical entity are used in a discovery logic; and
a discovery model comprising a specification of how the knowledge model and the logic model are assembled at run time of the discovery logic.

2. The signal-bearing medium of claim 1, wherein the knowledge Model comprises an object-oriented representation of the biological or chemical entity.

3. The signal-bearing medium of claim 2, wherein the knowledge model further comprises biological or chemical entity is represented using a Thing, a Key, a Fact, and a Relation.

4. The signal-bearing medium of claim 1, wherein the knowledge model is used to store the biological or chemical entity in a user-customizable database.

5. The signal-bearing medium of claim 1, wherein the logic model comprises an object-oriented representation of inference rules and wrappers around an algorithm.

6. The signal-bearing medium of claim 5, wherein the logic model is designed using at least one element selected from the group consisting of an event, an event handler and relation function.

7. The signal-bearing medium of claim 1, wherein the discovery model comprises the design and execution of an application.

8. The signal-bearing medium of claim 7, wherein the discovery model further comprises a kernel, a session, and an application.

9. The method of claim 1, wherein the knowledge model, the discovery model, and the logic model are designed using a knowledge oriented programming environment.

9. A method for maintaining biological and chemical knowledge, the method comprising:
translating data from biological and chemical data into a set of one or more components in a knowledge oriented programming environment;
translating algorithms and computational tools into the set of one or more components in a knowledge oriented programming environment; and
assembling a customized application for execution at run time from the set of one or more components.

10. The method of claim 9 further comprising providing a graphical user interface for accessing and designing a set of tools in a knowledge oriented programming environment and for executing the application.

11. A computerized system comprising:
a CPU;
a memory;
an knowledge oriented programming environment stored in the memory and executed by the CPU, the knowledge oriented programming environment comprising:
a knowledge model comprising a specification of how a biological or a chemical entity is represented;
a logic model comprising a specification of how a set of one or more algorithms associated with the biological or chemical entity are used in a discovery logic; and
a discovery model comprising a specification of how the knowledge model and the logic model are assembled at run time of the discovery logic.

12. The computerized system of claim 11, further comprising a graphical user interface for maintaining the knowledge model, the logic model, and the discovery model.
